(19) United States
(12) Patent Application Publication
(10) Pub. No.: US 2008/0077076 A1
(43) Pub. Date: Mar. 27, 2008

(54) IONTOPHORESIS DEVICE AND METHOD FOR OPERATION WITH A USB (UNIVERSAL SERIAL BUS) POWER SOURCE

(75) Inventor: Darrick Carter, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: TRANSCUTANEOUS TECHNOLOGIES INC., Tokyo (JP)

(21) Appl. No.: 11/847,092

(22) Filed: Aug. 29, 2007

Related U.S. Application Data
(60) Provisional application No. 60/823,890, filed on Aug. 29, 2006.

(51) Int. Cl. A61N 1/30

(52) U.S. Cl. 604/20

(57) ABSTRACT

An iontophoresis device includes an active agent reservoir that stores a quantity of an active agent for delivery into a biological interface. An active electrode element is operable to apply an electric potential to deliver at least a portion of the active agent into the biological interface. A port is selectively coupled to a computing system to provide both information and power to the iontophoresis device.

### CROSS-REFERENCE TO RELATED APPLICATION

This application claims the benefit under 35 U.S.C. § 119(e) of U.S. Provisional Patent Application No. 60/823,890 filed Aug. 29, 2006, which this provisional application is incorporated herein by reference in its entirety.

### BACKGROUND

1. Field

This disclosure generally relates to the field of iontophoresis, and more particularly to the controlling and/or monitoring of the delivery of an active agent, such as therapeutic agents or drugs, to a biological interface under the influence of electromotive force.

2. Description of the Related Art

Iontophoresis employs an electromotive force and/or current to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a power source, for example a chemical battery. Each electrode assembly typically includes a respective electrode element to apply an electromotive force and/or current. Such electrode elements often comprise a sacrificial element or compound, for example silver or silver chloride.

The active agent, such as a therapeutic agent, diagnostic agent, or pharmaceutical drug, may be cationic, anionic, or a mixture of such, and the power source can be configured to apply the appropriate voltage polarity based on the polarity of the active agent. Iontophoresis may be advantageously used to enhance or control the delivery rate of the active agent. As discussed in U.S. Pat. No. 5,395,310, the active agent may be stored in a reservoir such as a cavity. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ions, i.e., that of a charged active agent, prevents the back flux of the oppositely charged ions from the skin or mucous membrane.

Iontophoretic delivery devices typically employ a power supply to provide electromotive force or current to deliver active agent. The power supply may take the form of a battery, fuel cell and/or ultra-capacitor housed within the device or alternatively may take the form of an external power supply connected to the device. Iontophoretic devices may also employ a controller to control active agent delivery. The controller typically takes the form of an on-board regulating circuit which controls the current and/or voltage applied to the electrode elements from the power source. Such regulating circuits are typically preprogrammed or preconfigured and do not provide sufficient control of the drug delivery. In such devices, output of drug delivery data is either non-existent or not easily integrated into a clinical record system, which may be accessed for example, via a medical practitioner's personal computer (PC). Alternatively, the controller may take the form of an external device, for example a PC (Personal Computer) to control the drug delivery. In such an embodiment, an iontophoretic power supply may be electrically coupled to the iontophoretic device via a pair of leads respectively connected to the active electrode and the counter electrode assemblies of the iontophoretic device. The iontophoretic power supply may be connected to the PC via a communications port to control the drug delivery. The power supply provides the power for drug delivery while the PC, which operates on a separate power supply, is used to provide commands to control the current or drug delivery. Thus, separate power sources are employed for drug delivery and for providing commands to control drug delivery of the iontophoretic device.

Commercial acceptance of iontophoresis devices is dependent on a variety of factors, such as cost to manufacture. Thus, it would be desirable to have an iontophoresis system that reduces the number of separate power sources and system components while providing easy access to drug delivery data and control of drug delivery.

### BRIEF SUMMARY OF THE INVENTION

According to one embodiment, an iontophoresis device may be summarized as comprising an active agent reservoir that stores a quantity of an active agent, an active electrode element operable to apply an electrical potential of a first polarity to deliver at least a portion of the active agent to a biological interface from the iontophoresis device, and a port selectively coupleable to a computing system to provide both information and power to the iontophoresis device.

According to one embodiment, the computing system is configured to monitor at least one operational aspect of the iontophoresis device via a communications path established between the iontophoresis device and the computing system. The communications path may be at least one of a wired network connection or wireless network connection.

According to another embodiment, an iontophoresis device may further include an iontophoretic interface communicatively coupled to the computing system and to the port, and operable to at least partially control the delivery of the active agent to the biological interface from the iontophoresis device and supply power to the iontophoresis device from the computer system, wherein the iontophoresis device is responsive to control signals provided from the iontophoretic interface via the port. At least some of the control signals provided by the iontophoretic interface to the iontophoresis device may be indicative of an amount of the active agent to deliver to a biological interface from the iontophoresis device, a current flow through at least a portion of the iontophoresis device and/or a voltage across at least a portion of the iontophoresis device.

According to another embodiment, the iontophoretic interface is configured to monitor at least one operational aspect of the iontophoresis device via the port, including an amount of the active agent delivered by the iontophoresis device, a current flow through at least a portion of the iontophoresis device and/or voltage across at least a portion of the iontophoresis device.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic illustration of an iontophoresis system having an iontophoresis device that is provided with information and power via a port, according to one illustrated embodiment.

FIG. 2 is a schematic illustration of an iontophoresis system having an iontophoresis device that is provided with information and power via a port, according to another illustrated embodiment.

FIG. 3 is a block diagram of an iontophoresis device comprising a port coupled to active and counter electrode assemblies, a controller, and a regulator, according to one illustrated embodiment.

FIG. 4 is a low level flow diagram of a method of operating the iontophoresis device to receive requests, in the form of control signals, for adjusting at least one operational parameter and modifying the active agent delivery in response thereto, according to one illustrated embodiment.

FIG. 5 is a low level flow diagram of a method of monitoring operational parameter information, according to one illustrated embodiment.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein and in the claims, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizeable and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opiod agonists; sumatriptan succinate, zolmitriptan, naratriptan HCl, rizatriptan benzoate, almotriptan malate, frovatriptan succinate and other 5-hydroxytryptamine 1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and a mixture thereof.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the claimed invention.

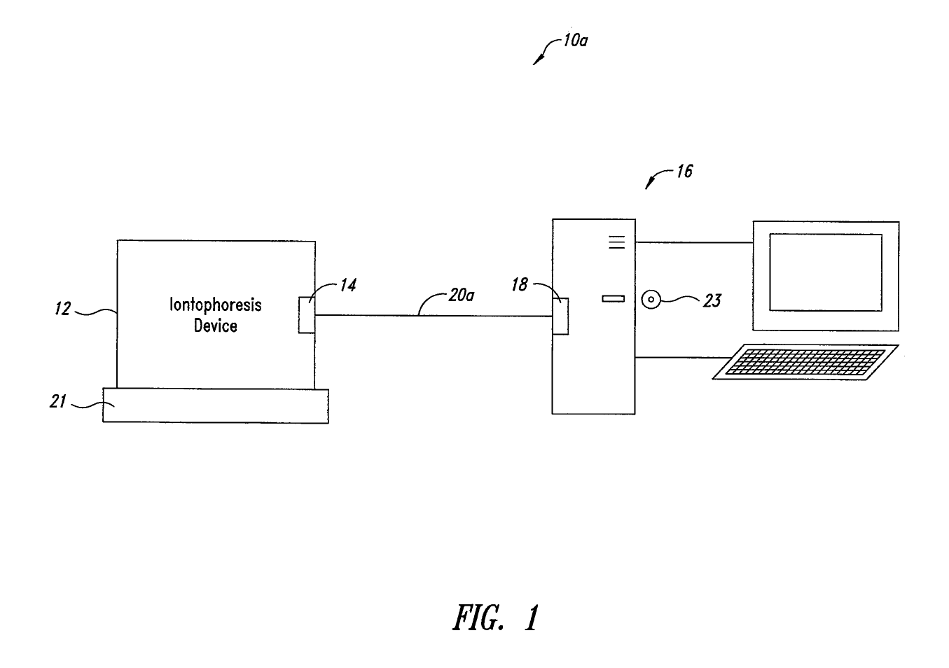

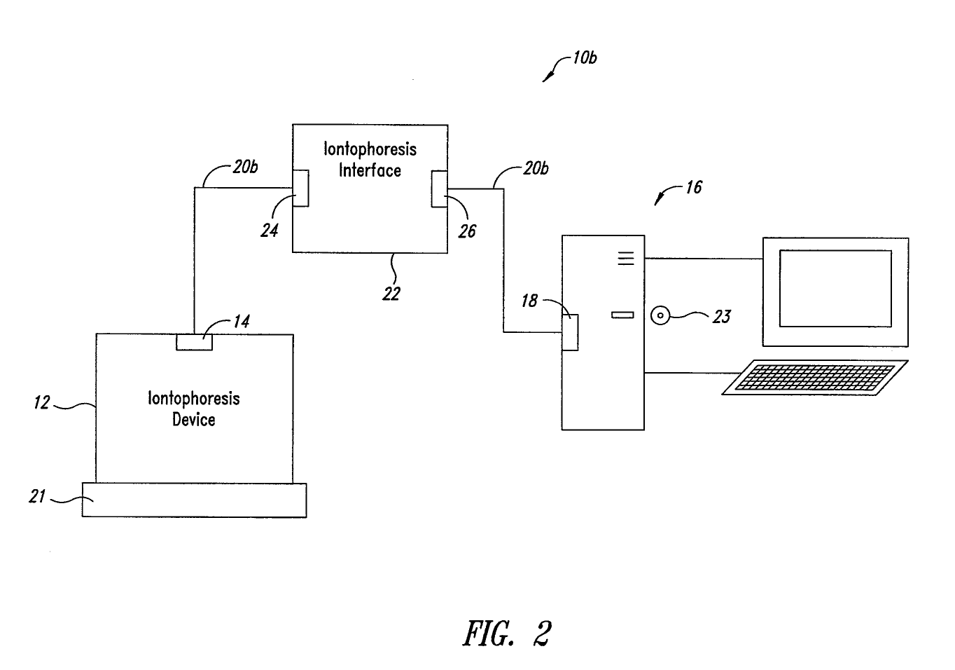

FIG. 1 shows a schematic illustration of an iontophoresis system 10a having an iontophoresis device 12 that is provided with information and power via a port 14, according to one illustrated embodiment while FIG. 2 shows a schematic illustration of an iontophoresis system 10b having the iontophoresis device 12 that is provided with information and power via the port 14, according to another illustrated embodiment.

The iontophoresis system 10a comprises the iontophoresis device 12 communicatively and electrically coupled to a computing system 16 via the port 14 of the iontophoresis device 12 and a host hub 18 that is located on a controller of the computing system 16. The port 14 is selectively coupleable to the host hub 18 to establish one or more paths 20a between the iontophoresis device 12 and the computing system 16. The paths 20a, which may be one of wired or wireless, allows for the transfer of power from the computing system 16 to the iontophoresis device 12 and the transmission of information therebetween.

The iontophoresis device 12, described in detail below, includes an active agent reservoir that stores a quantity of an active agent for delivery into a biological interface 21. The port 14 of the iontophoresis device 12 may be one or more USB (Universal Serial Bus) connectors or one or more connectors having embedded wires, pins or conduits with the capacity to supply sufficient electrical power to operate the iontophoresis device 12 and further embedded wires, pins or conduits serving as a medium for sending data and commands.

The computing system 16 may comprise, for example, a PC (Personal Computer), laptop, hand-held computer, PDA (Personal Digital Assistant), BLACKBERRY® or any other processor-based system. The computing system 16 is operable to provide information to the iontophoresis device 12 from the host hub 18 in the form of control signals. The control signals at least partially control the delivery of the active agent into the biological interface 21 from the iontophoresis device 12. The iontophoresis device 12 is responsive to the control signals provided from the computing system 16 via the communications path 20a. Additionally or in parallel to the control signals, the computing system 16 supplies power to the iontophoresis device 12 via the communications path 20a. The supplied power is transferred from the host hub 18 to the port 14. In response to the control signals being sent from the computing system 16, the iontophoresis device 12 may send response signals to the computing system 16 via the port 14 and the host hub 18.

In another embodiment, as illustrated in FIG. 2, the iontophoresis system 10b comprises an iontophoretic interface 22 communicatively and electrically coupled to the host hub 18 of the computing system 16 and to the port 14 of the iontophoresis device 12. One or more paths 20b are established between the port 14 and a first iontophoretic interface port 24, and between a second iontophoretic interface port 26 and the host hub 18. The one or more paths 20b, may be one of wired or wireless, allows for the transfer of power from the computing system 16 to the iontophoresis device 12 and the transmission of information therebetween.

The iontophoretic interface 22 is operable to provide information to the iontophoresis device 12 from the first iontophoretic interface port 24 in the form of control signals. The control signals at least partially control the delivery of the active agent into the biological interface 21. The control signals may originate from the computing system 16 or from the iontophoretic interface 22. For example, the iontophoretic interface 22 may comprise a computer readable medium 23 (e.g., floppy disk, compact disk, memory stick, etc.) having code stored therein that permits the iontophoretic interface 22 to control the active agent delivery. The iontophoresis device 12 is responsive to the control signals provided from the iontophoretic interface 22 via the paths 20b.

Additionally or in parallel to the control signals, the computing system 16 supplies power to the iontophoretic interface 22 and to the iontophoresis device 12 via the one or more paths 20b. The power is transferred from the host hub 18 to the second iontophoretic port 26 to power the iontophoretic interface 22, and from the first iontophoretic port 24 to the port 14 to power the iontophoresis device 12.

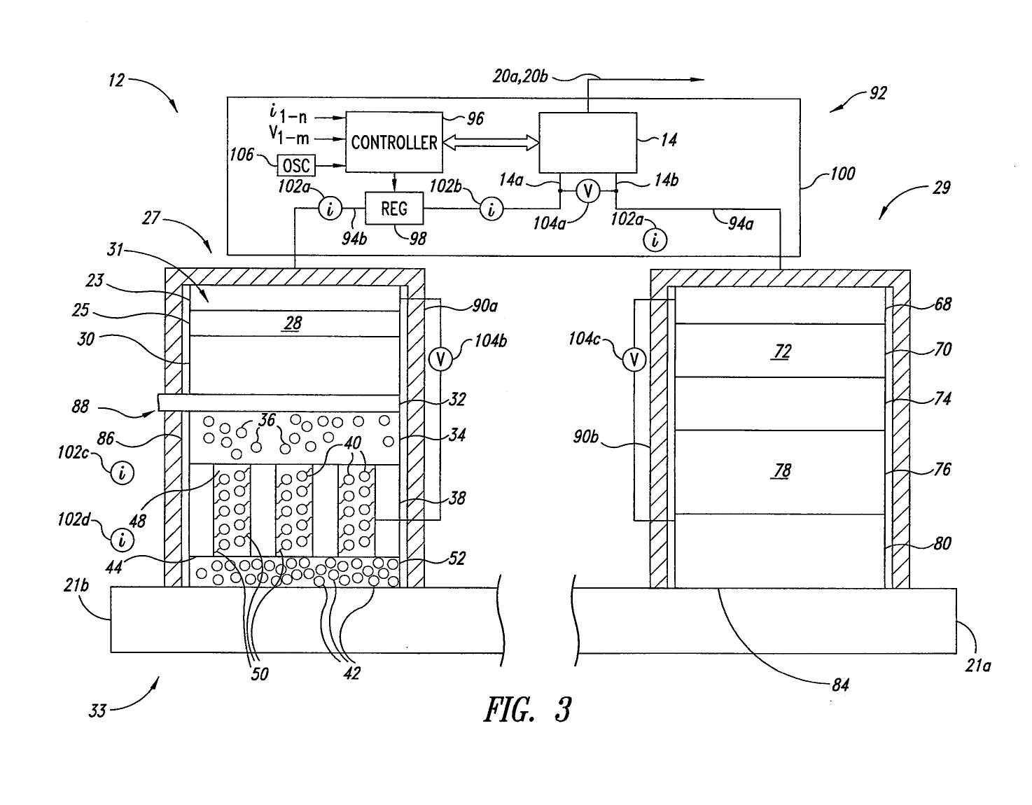

FIG. 3 shows the iontophoresis device 12, comprising: an active electrode assembly 27 positioned on or proximate a first portion 21b of a biological interface 21, and counter assembly 29 positioned proximate a second portion 21a of the biological interface 21, each electrode assembly 27, 29 electrically coupled to the port 14 and operable to supply at least one active agent to the second portion 21b of the biological interface 21 via iontophoresis, according to one illustrated embodiment. As noted above, the biological interface 21 may take a variety of forms, for example, a portion of skin, mucous membrane, gum, tooth or other tissue.

In the illustrated embodiment, the active electrode assembly 27 comprises, from an interior 31 to an exterior 33 of the active electrode assembly 27, an active electrode element 23, an electrolyte reservoir 25 storing an electrolyte 28, an inner ion selective membrane 30, an optional inner sealing liner 32, an inner active agent reservoir 34 storing active agent 36, an outermost ion selective membrane 38 that caches additional active agent 40, and further active agent 42 carried by an outer surface 44 of the outermost ion selective membrane 38. Each of the above elements or structures will be discussed in detail below.

The active electrode element 23 is coupled to a first line 14a of the port 14 and positioned in the active electrode assembly 27 to apply an electromotive force or current to transport active agent 36, 40, 42 via various other components of the active electrode assembly 27. A counter electrode element 68 is coupled to a second line 14b of the port 14. The active electrode element 23 may take a variety of forms. For example, the active electrode element 23 may include a sacrificial element, for example a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a carbon-based active electrode element 23. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The electrolyte reservoir 25 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 25 may take the form of a pouch or other receptacle, a membrane with pores, cavities or interstices, particularly where the electrolyte 28 is a liquid.

The electrolyte 28 may provide ions or donate charges to prevent or inhibit the formation of gas bubbles (e.g., hydrogen) on the active electrode element 23 in order to enhance efficiency and/or increase delivery rates. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18. As discussed further below, in some embodiments the electrolyte 28 may provide or donate ions to substitute for the active agent, for example substituting for the active agent 40 cached in the outermost ion selective membrane 39. Such may facilitate transfer of the active agent 40 to the biological interface 18, for example, increasing and/or stabilizing delivery rates. A suitable electrolyte may take the form of a solution of 0.5M disodium fumarate: 0.5M Poly acrylic acid (5:1).

The inner ion selective membrane 30 is generally positioned to separate the electrolyte 28 and the inner active agent reservoir 34. The inner ion selective membrane 30 may take the form of a charge selective membrane. For example, where the active agent 36, 40, 42 comprises a cationic active agent, the inner ion selective membrane 38 may take the form of an anion exchange membrane, selective to substantially pass anions and substantially block cations. Also, for example, where the active agent 36, 40, 42 comprises an anionic active agent, the inner ion selective membrane 38 may take the form of an cationic exchange membrane, selective to substantially pass cations and substantially block anions. The inner ion selective membrane 38 may advantageously prevent transfer of undesirable elements or compounds between the electrolyte 28 and the active agents 26, 40, 42. For example, the inner ion selective membrane 38 may prevent or inhibit the transfer of hydrogen (H⁺) or sodium (Na⁺) ions from the electrolyte 72, which may increase the transfer rate and/or biological compatibility of the iontophoresis device 10.

The optional inner sealing liner 32 separates the active agent 36, 40, 42 from the electrolyte 28 and is selectively removable via slot or opening 88. The inner sealing liner 32 may advantageously prevent migration or diffusion between the active agent 36, 40, 42 and the electrolyte 28, for example, during storage.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36, and in some embodiments may even be the active agent 36 itself, for example, where the active agent 36 is in a gel, semi-solid or solid form. For example, the inner active agent reservoir 34 may take the form of a pouch or other receptacle, a membrane with pores, cavities or interstices, particularly where the active agent 36 is a liquid. The inner active agent reservoir 34 may advantageously allow larger doses of the active agent 36 to be loaded in the active electrode assembly 27.

The outermost ion selective membrane 38 is positioned generally opposed across the active electrode assembly 27 from the active electrode element 23. The outermost membrane 38 may, as in the embodiment illustrated in FIG. 3 take the form of an ion exchange membrane, pores 48 (only one called out in FIG. 3 for sake of clarity of illustration) of the ion selective membrane 38 including ion exchange material or groups 50 (only three called out in FIG. 3 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., strontium, lidocaine), the outermost ion selective membrane 38 may take the form of a cation exchange membrane. Alternatively, where the active agent 36, 40, 42 is an anion (e.g., fluoride), the outermost ion selective membrane 38 may take the form of an anion exchange membrane.

The outermost ion selective membrane 38 may advantageously cache active agent 40. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing a removably releasable outer release liner to retain the active agent 40 until the outer release liner is removed prior to use.

The outermost ion selective membrane 38 may be preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 27, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

An interface coupling medium (not shown) may be employed between the electrode assembly and the biological interface 21. The interface coupling medium may, for example, take the form of an adhesive and/or gel. The gel may, for example, take the form of a hydrating gel.

According to one embodiment, as described above, the port 14 may take the form of a USB connector that is electrically and communicatively coupled to the computing system 16 via the one or more paths 20a, 20b. The paths 20a, 20b allow for the transfer of both power and information from the computing system 16 and/or the iontophoretic interface 22 to the iontophoresis device 12. The computing system may, for example, provide a voltage of 12.8V DC, with tolerance of 0.8V DC, and a current of 0.3 mA via the port 14. The first and second lines 14a, 14b of the port may be coupled to the active and counter electrode elements 23, 68, respectively. The port 14 may be selectively electrically coupled to the active and counter electrode assemblies 27, 29 via a control circuit 92 (discussed below), for example, via carbon fiber ribbons 94a, 94b or any other suitable wiring. The iontophoresis device 12 may include a controller 96 and a regulating circuit 98 (discussed below) formed from discrete and/or integrated circuit elements to control and/or monitor operation, and/or regulate the voltage, current and/or power delivered to the electrode assemblies 27, 29. For example, the iontophoresis device 12 may include a diode to provide a constant current to the electrode elements 31, 68.

As suggested above, the active agent 42 may take the form of a cationic or an anionic drug or other therapeutic agent. Consequently, the first and second lines 14a, 14b or terminals of the port 14 may be reversed. Likewise, the selectivity of the outermost ion selective membranes 38, 80 and inner ion selective membranes 30, 74 may be reversed.

The control circuit 92 includes the controller 96 and regulating circuit 98, which may be mounted or carried by a circuit board, such as flexible circuit board 100. The flexible circuit board 100 may comprise one or more insulative layers, and may optionally comprise one or more conductive layers interlaced with the insulative layers. The circuit board 100 may form one or more vias, to make electrically couplings between the surfaces of the circuit board 100 and/or between various ones of the conductive layers.

The control circuit 92 may also include one or more current sensors 102a-102d (collectively 102), positioned and configured to sense or measure current through one or more reservoirs, membranes or other structures. The control circuit 92 may also include one or more voltage sensors 104a-104c (collectively 104), positioned and configured to sense or measure voltage across one or more reservoirs, membranes or other structures. The current and voltage sensors 102, 104 provide signals indicative of the current i₁-iₙ, and signals indicative of the voltage v₁-vₘ, respectively, to the controller 96.

The control circuit 92 may also include an off-chip oscillator 106 that provides a frequency signal to the controller 96 to form a clock signal. Alternatively, the controller 92 may employ an on-chip oscillator.

The controller 92 may employ the signals indicative of the current i₁-iₙ, and signals indicative of the voltage v₁-vₘ, as well as the frequency signals to analyze operation of the device, and to produce additional performance information, as discussed in more detail below.

The controller 92 is communicatively coupled to receive and/or provide information from and/or to the port 14. Thus, the controller 92 may cause the port 14 to transmit parameter and/or performance information from the iontophoresis device 12 through the one or more paths 20a, 20b. The controller 92 may cause the port 14 to transmit the parameter and/or performance information in response to a request from the computing system 16 and/or the iontophoretic interface 22. The controller 92 may receive the request in the form of control signals from the computing system 16 and/or the iontophoretic interface 22 via the port 14. The control signals may be indicative of a request for monitoring and/or adjusting at least one operational aspect of the iontophoresis device 12.

The controller 96 may use the parameter and/or other performance information that it generates, as well as parameters and/or other performance information requests received from the computing system 16 and/or the iontophoretic interface 22 to modify the active agent delivery regime. For example, the controller 96 may implement a new or updated active agent delivery regime based on the control signals received from the computer system 16 and/or the iontophoretic interface 22. The control signals are indicative of the desired parameters and/or other performance information for active agent delivery. The controller 96 provides appropriate control signals to the regulating circuit 98 to implement the new or revised regime. The regulating circuit 98 may take the form a voltage control regulator and/or current control regulator, that controls the delivery of active agent by controlling voltage applied across, or current applied to, the electrode elements 23, 68. Additionally or in parallel to modifying the active agent delivery regime, the computer system 16 supplies sufficient power via the one or more paths 20a, 20b to operate the iontophoresis device 12.

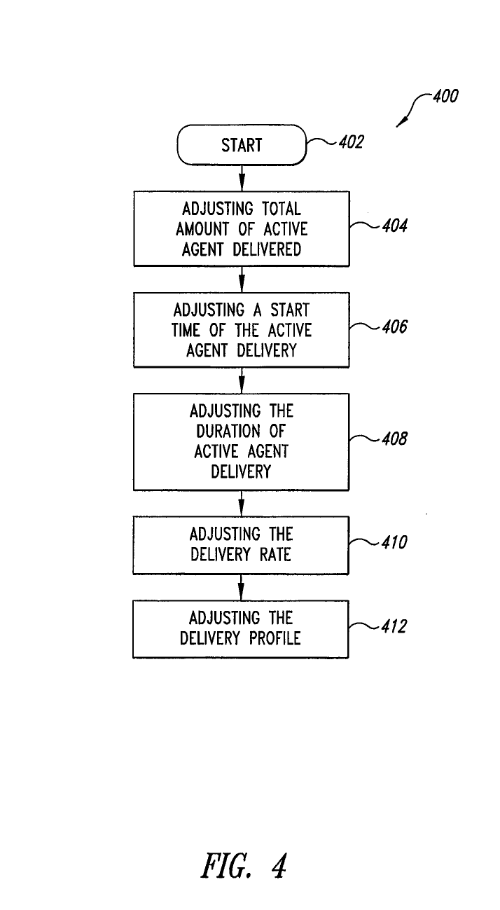

FIG. 4 shows a low level flow diagram of a method 400 of operating the iontophoresis device 12 to receive requests, in the form of control signals, for adjusting at least one operational parameter and modifying the active agent delivery in response thereto, according to one illustrated embodiment. The method may be implemented by the controller 96, as either software or firmware instructions, or as hardwired logic.

The method 400 starts at 402. For example, the method 400 may start in response to control signals received from the computer system 16 and/or the iontophoretic interface 22, and may run in parallel with the transfer of power from the computing system 16 to the iontophoresis device 12 via the one or more paths 20a, 20b. The control signals at least partially control the delivery of the active agent

At 404, the controller 96 adjusts a total amount of active agent delivered. For example, the controller 96 may adjust a current through a reservoir, membrane or other structure, and/or may adjust a voltage across a reservoir, membrane or other structure to modify the total amount of active agent delivered. For instance, the controller 96 may modify the amount of current drawn over an entire period of time during which the active agent is delivered, and determine the amount of active agent delivery based on a defined relationship between current and rate of active agent delivery, based on the knowledge of the total time of delivery. Such may be refined using empirically derived relationships.

At 406, the controller 96 adjusts a time at which a delivery of the active agent starts. For example, the controller 96 may start a timer or clock when current beings to flow, for example in response to activation of a switch.

At 408, the controller 96 modifies a duration during which the active agent is delivered. For example, the controller 96 may stop a timer or clock when current stops flowing, for example in response to deactivation of a switch.

At 410, the controller 96 modifies a rate at which the active agent is delivered. For example, the controller 96 may modify a current through a reservoir, membrane or other structure, and/or may modify a voltage across a reservoir, membrane or other structure to modify the rate at which the active agent is delivered. For instance, the controller 96 may modify an instantaneous rate based on a relationship between current and rate of delivery and a knowledge of the instantaneous current. Also for instance, the controller 96 may modify an average rate by cumulating or integrating the modified instantaneous rates.

At 412, the controller 96 modifies a delivery profile at which the active agent is delivered. For example, the controller 96 may modify a current through a reservoir, membrane or other structure, and/or may modify a voltage across a reservoir, membrane or other structure to modify the total amount of active agent delivered. For instance, the controller 96 may modify the current over time, determining the delivery profile based at least in part on a relationship between the current and rate of delivery, and a knowledge of the instantaneous current through the active agent delivery. Such may be refined using empirically derived relationships, for example, a relationship between rate of delivery and voltage, a relationship between rate of delivery and impedance where impedance is modified by adjusting another monitored parameter (e.g., current or voltage).

The method 400 may include additional acts, may omit some of the above-described acts and/or may perform acts in a different order than set out in the flow diagram.

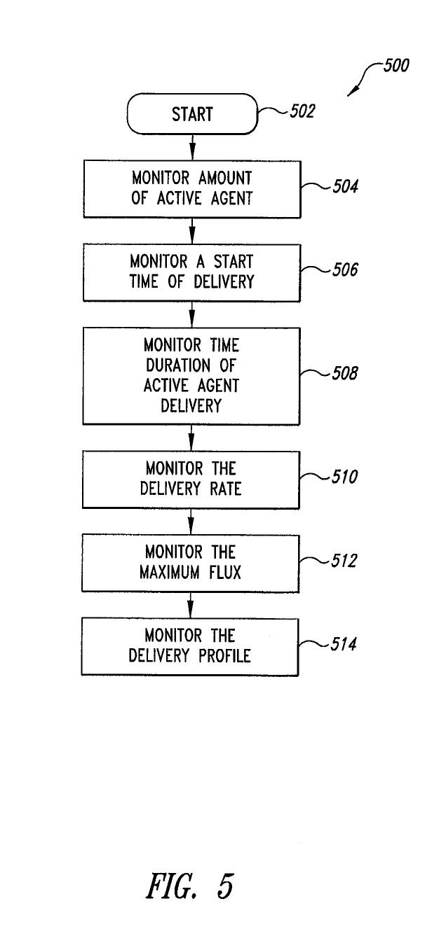

FIG. 5 shows a low level flow diagram of a method 500 of monitoring operational parameter information, according to one illustrated embodiment. The monitored operational parameter information may be sent to the computer system 16 and/or the iontophoretic interface 22 via the one or more paths 20a, 20b. The method 500 may be implemented by the controller 96, as either software or firmware instructions, or as hardwired logic.

The method 500 starts at 502. For example, the method 500 may start in response to control signals received from the computer system 16 and/or the iontophoretic interface 22 or in response to an activation of the iontophoresis device 12. Activation may be the closing of a switch, or simply the application of the iontophoresis device 12 to the biological interface 21 that completes the circuit. The method may run in parallel with the transfer of power from the computing system 16 to the iontophoresis device 12 via the one or more paths 20a, 20b.

At 504, the controller 96 monitors a total amount of active agent delivered. For example, the controller 96 may monitor a current through a reservoir, membrane or other structure, and/or may monitor a voltage across a reservoir, membrane or other structure to determine the total amount of active agent delivered. For instance, the controller 96 may monitor the amount of current drawn over an entire period of time during which the active agent is delivered, and determine the amount of active agent delivery based on a defined relationship between current and rate of active agent delivery, based on the knowledge of the total time of delivery. Such may be refined using empirically derived relationships.

At 506, the controller 96 monitors a time at which a delivery of the active agent starts. For example, the controller 96 may start a timer or clock when current beings to flow, for example in response to activation of a switch or simply the completion of the circuit by the placement of the iontophoresis device 12 on the biological interface 21. Alternatively, the timer or clock may start in response to the control signals received from the computer system 16 and/or iontophoretic interface 22.

At 508, the controller 96 monitors a duration during which the active agent is delivered. For example, the controller 96 may stop a timer or clock when current stops flowing, for example in response to deactivation of a switch or simply the opening of the circuit path between the electrode assemblies 27, 29 by the removal of the iontophoresis device 12 from the biological interface 21.

At 510, the controller 96 monitors a rate at which the active agent is delivered. For example, the controller 96 may monitor a current through a reservoir, membrane or other structure, and/or may monitor a voltage across a reservoir, membrane or other structure to determine the rate at which the active agent is delivered. For instance, the controller 96 may monitor an instantaneous rate based on a relationship between current and rate of delivery and a knowledge of the instantaneous current. Also for instance, the controller 96 may monitor an average rate by cumulating or integrated the instantaneous rates.

At 512, the controller 96 monitors a maximum flux at which the active agent is delivered. For example, the controller 96 may monitor a current through a reservoir, membrane or other structure, and/or may monitor a voltage across a reservoir, membrane or other structure to determine the maximum flux at which the active agent is delivered. For instance, the controller 96 may monitor the maximum current draw. The controller 96 may determine the maximum flux based on a relationship between current and rate of delivery, and a knowledge of the maximum current draw.

At 514, the controller 96 monitors a delivery profile at which the active agent is delivered. For example, the controller 96 may monitor a current through a reservoir, membrane or other structure, and/or may monitor a voltage across a reservoir, membrane or other structure to determine the total amount of active agent delivered. For instance, the controller 96 may monitor the current over time, determining the delivery profile based at least in part on a relationship between current and rate of delivery, and a knowledge of the instantaneous current through the active agent delivery. Such may be refined using empirically derived relationships, for example, a relationship between rate of delivery and voltage, a relationship between rate of delivery and impedance where impedance is either monitored or determined from another monitored parameter (e.g., current or voltage).

The method 500 may include additional acts, may omit some of the above-described acts and/or may perform acts in a different order than set out in the flow diagram.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

I/We claim:

1. An iontophoresis device, comprising:
an active agent reservoir that stores a quantity of an active agent;
an active electrode element operable to apply an electrical potential of a first polarity to deliver at least a portion of the active agent to a biological interface from the iontophoresis device; and
a port selectively coupleable to a computing system to provide both information and power to the iontophoresis device.

2. The iontophoresis device of claim 1 wherein the computing system is operable to provide information in the form of control signals to at least partially control the delivery of the active agent to the biological interface from the iontophoresis device and to supply power to the iontophoresis device, wherein the iontophoresis device is responsive to the control signals provided from the computing system via the port.

3. The iontophoresis device of claim 2 wherein at least some of the control signals provided by the computing system to the iontophoresis device are indicative of a delivery rate for delivering active agent to the biological interface from the iontophoresis device.

4. The iontophoresis device of claim 2 wherein at least some of the control signals provided by the computing system to the iontophoresis device are indicative of an amount of the active agent to deliver to a biological interface from the iontophoresis device.

5. The iontophoresis device of claim 2 wherein at least some of the control signals provided by the computing system to the iontophoresis device are indicative of a current flow through at least a portion of the iontophoresis device.

6. The iontophoresis device of claim 2 wherein at least some of the control signals provided by the computing system to the iontophoresis device are indicative of a voltage across at least a portion of the iontophoresis device.

7. The iontophoresis device of claim 2 wherein at least some of the control signals provided by the computing system to the iontophoresis device are indicative of a time during which the iontophoresis device is to actively deliver the active agent to the biological interface.

8. The iontophoresis device of claim 2 wherein at least some of the control signals provided by the computing system to the iontophoresis device are indicative of a delivery profile for delivering the active agent from the iontophoresis device to the biological interface.

9. The iontophoresis device of claim 2 wherein the computing system is configured to monitor at least one operational aspect of the iontophoresis device via a communications path established between the iontophoresis device and the computing system.

10. The iontophoresis device of claim 9 wherein the computing system monitors a rate of delivery of the active agent.

11. The iontophoresis device of claim 9 wherein the computing system monitors an amount of the active agent delivered by the iontophoresis device.

12. The iontophoresis device of claim 9 wherein the computing system monitors a current flow through at least a portion of the iontophoresis device.

13. The iontophoresis device of claim 9 wherein the computing system monitors voltage across at least a portion of the iontophoresis device.

14. The iontophoresis device of claim 9 wherein the computing system monitors a time during which the iontophoresis device has been at least one of active or inactive.

15. The iontophoresis device of claim 9 wherein the computing system monitors a delivery profile of the active agent over a period of time.

16. The iontophoresis device of claim 9 wherein the port comprises at least one Universal Serial Bus (USB) connector.

17. The iontophoresis device of claim 1, further comprising:
an iontophoretic interface communicatively coupled to the computing system and to the port, and operable to at least partially control the delivery of the active agent to the biological interface from the iontophoresis device and supply power to the iontophoresis device from the computer system, wherein the iontophoresis device is responsive to control signals provided from the iontophoretic interface via the port.

18. The iontophoresis device of claim 17 wherein at least some of the control signals provided by the iontophoretic interface to the iontophoresis device are indicative of a delivery rate for delivering active agent to the biological interface from the iontophoresis device.

19. The iontophoresis device of claim 17 wherein at least some of the control signals provided by the iontophoretic interface to the iontophoresis device are indicative of a time during which the iontophoresis device is to actively deliver the active agent to the biological interface.

20. The iontophoresis device of claim 17 wherein at least some of the control signals provided by the iontophoretic interface to the iontophoresis device are indicative of a delivery profile for delivering the active agent from the iontophoresis device to the biological interface.

21. The iontophoresis device of claim 17 wherein the iontophoretic interface is configured to monitor at least one operational aspect of the iontophoresis device via the port.

22. The iontophoresis device of claim 21 wherein the iontophoretic interface monitors a rate of delivery of the active agent.

23. The iontophoresis device of claim 21 wherein the iontophoretic interface monitors a time during which the iontophoresis device has been at least one of active or inactive.

24. The iontophoresis device of claim 21 wherein the iontophoretic interface monitors a delivery profile of the active agent over a period of time.

25. The iontophoresis device of claim 21 wherein the port comprises at least one Universal Serial Bus (USB) connector.
